(19) United States
(12) Patent Application Publication
(10) Pub. No.: US 2009/0155223 A1
(43) Pub. Date: Jun. 18, 2009

(54) CELL-BASED COMPOSITIONS AND METHODS FOR TREATING CONDITIONS OF THE NERVOUS SYSTEM

(75) Inventors: Douglas A. Kerr, Ruxton, MD (US); Michael Gorelik, Reisterstown, MD (US); Michael Levy, Phoenix, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 12/192,051

(22) Filed: Aug. 14, 2008

Related U.S. Application Data
(60) Provisional application No. 60/964,643, filed on Aug. 14, 2007.
(60) Provisional application No. 61/133,333, filed on Jun. 28, 2008.

(51) Int. Cl. A61K 45/00; A61P 25/28

(52) U.S. Cl. 424/93.7

(57) ABSTRACT

Disclosed herein are cell-based compositions for the treatment of conditions of the nervous system and methods for their use. In one embodiment, a cell-based composition comprises glial-restricted progenitors (GRPs) genetically modified to express a targeting ligand on their cell surface. Methods for the preparation of such cell-based compositions are disclosed. Also disclosed is a method for treating a subject suffering from a condition of the central nervous system by providing therapeutic cells (e.g., GRPs) through an intra-arterial route of administration.

### CROSS-REFERENCE

This application claims the benefit of U.S. Provisional Application Ser. No. 61/133,333, filed Jun. 28, 2008, and U.S. Provisional Application Ser. No. 60/964,643, filed on Aug. 14, 2007, the contents of both of which are incorporated by reference herein in their entirety.

### BACKGROUND OF THE INVENTION

One of the most promising therapeutic approaches for treating many pathological conditions in the nervous system, e.g., multiple sclerosis, Alzheimer's disease, and Parkinson's disease, is cell replacement with transplanted therapeutic cells. However, despite its promise, this approach still faces a number of formidable technical hurdles.

### SUMMARY OF THE INVENTION

Disclosed herein are cell-based compositions for the treatment of conditions of the nervous system, e.g., a CNS condition, and methods for their use.

Accordingly in one aspect provided herein is a method for treating a demyelinating condition by administering to a subject in need thereof a plurality of transplantation-competent GRPs, in which (i) the transplantation-competent GRPs were produced by contacting a plurality of GRPs with an agent that induces differentiation of GRPs into oligodendrocytes; or (ii) are administered through an intra-arterial route. In some embodiments, the transplantation-competent GRPs overexpress VLA-4 on their surface. In some embodiments, the expression of VLA-4 is inducible or repressible. In some embodiments, the transplantation-competent GRPs overexpress chemokine receptors on their surface to enhance migration into the CNS.

In another aspect provided herein is a method for delivering GRPs to the central nervous system of a subject in need thereof, comprising administering to the subject a plurality of GRPs through an intra-arterial route. In some embodiments, the subject to be treated is suffering from a demyelinating disease (e.g., multiple sclerosis or transverse myelitis) or a neurodegenerative disease. In some embodiments, the demyelinating disease comprises an inflammatory condition.

In some embodiments the plurality of GRPs to be administered to the subject contains GRPs that overexpress a polypeptide comprising the amino acid sequence of aVLA-4 subunit on their surface. In some embodiments, the administered GRPs myelinate neurons in the CNS of the subject.

In a further aspect provided herein is a composition comprising a genetically modified GRP (e.g., a human recombinant GRP) comprising one or more expression vectors for expression of VLA-4, in which the composition is pharmaceutically acceptable for intra-arterial delivery to the central nervous system of a subject in need thereof. In some embodiments, the expression of VLA-4 from the one or more expression vectors is inducible or repressible. In some embodiments, the genetically modified GRP in this composition was obtained by a method comprising differentiation of an embryonic stem cell. In some embodiments, the genetically modified GRP in this composition further comprises an expression vector for a reporter protein (e.g., a fluorescent protein, an enzyme, or a poly-Lysine-containing reporter protein). In one embodiment, where the reporter protein is a fluorescent protein, the reporter protein emits green fluorescence (e.g., a green fluorescent protein), yellow fluorescence (e.g., a yellow fluorescent protein), or red fluorescence (e.g., DS-Red). In one embodiment, where the reporter protein is an enzyme, the enzyme is β-galactosidase, herpes thymidine kinase, or luciferase. In some embodiments, where the reporter protein is a poly-Lysine-containing reporter protein, the reporter protein comprises about 50 to about 250 lysines

In a related aspect provided herein is a GRP (e.g., a human GRP) comprising an exogenous VCAM-1 ligand on the cell surface or an exogenous nucleic acid encoding (a) VCAM-1 ligand or (b) a chemokine receptor. In some embodiments, the exogenous VCAM-1 ligand is a polypeptide comprising: (i) an amino acid sequence at least 85% identical to the amino acid sequence of any of human, rodent, or canine Integrin α4, Integrin α9, Integrin β1, Integrin β7, Integrin αD, Ezrin, Moesin, VCAM-1, and Cathepsin G; or (ii) a heavy chain or light chain of antibody that binds specifically to human, rodent, or canine VCAM-1. In one embodiment, the exogenous VCAM-1 ligand comprises a polypeptide comprising the amino acid sequence of human, rodent, or canine Integrin α4 or Integrin β1. In some embodiments, the above-mentioned exogenous nucleic acid also includes a promoter operably linked to the open reading frame for the VCAM-1 ligand or chemokine receptor. In one embodiment, the promoter is an inducible promoter. In some embodiments, the GRP also comprises a detectable label (e.g., a detectable label that is detected in the CNS by a non-invasive method). In one embodiment, where the GRP contains a detectable label, the GRP comprises an exogenous nucleic acid encoding a reporter protein (e.g., a fluorescent protein, an enzyme, or a poly-lysine reporter protein) that, when expressed, provides the detectable label. In some embodiments, the detectable label in the GRP comprises one or more nanoparticles, e.g., (a fluorescent nanoparticle such as a Q-Dot, an iron oxide nanoparticle, or a liquid perfluorocarbon nanoparticle)

In a further aspect provided herein is a method for treating a CNS condition, comprising administering to a subject in need thereof a substantially pure population of therapeutic cells expressing an exogenous VCAM-1 ligand by an intra-arterial route. In some embodiments, the therapeutic cells are therapeutic cells committed to a neuronal cell fate (e.g., neural stem cells or neurons) or glial cell fate (e.g., GRPs or oligodendrocytes). In some embodiments, the exogenous VCAM-1 ligand comprises an amino acid sequence at least 85% identical to the amino acid sequence of a VLA-4 subunit (e.g., Integrin α4 or Integrin β1). In one embodiment, the CNS condition to be treated is a demyelinating condition

In yet another aspect provided herein is a method for detecting therapeutic cells in the CNS, comprising imaging, by a non-invasive imaging technique, a region of the CNS of a subject administered the therapeutic cells by an intra-arterial route, the therapeutic cells being detectably labeled for detection by the non-invasive imaging technique and expressing an exogenous VCAM-1 ligand

In a further aspect provided herein is a method for treating a CNS condition, comprising dispersing a plurality of therapeutic cells to a plurality of separate brain regions (e.g., neocortex, hippocampus, or nuclei of the basal ganglia) in a subject in need thereof. In some embodiments, the brain regions to which the therapeutic cells are dispersed are separated by a distance of about 0.05% to about 50% of the width, length, or height of the subject's brain. In some embodiments, dispersing the cells does not include perforating the subject's skull or skin on the subject's head.

In another aspect provided herein is a device comprising a needle for intra-arterial administration, a container in fluid communication with the needle, and within the container, a plurality of transplantation-competent GRPs and a pharmaceutically-acceptable carrier

### INCORPORATION BY REFERENCE

All publications, patents, and patent applications mentioned in this specification are herein incorporated by reference for the purpose for which they have been cited herein.

### BRIEF DESCRIPTION OF THE DRAWINGS

FIG. 1 shows an illustrative western blot analysis of rat GRP expression of 2′,3′-cyclic nucleotide-3′-phosphodiesterase 1 and 2 (CNPase1 and 2) in rat GRPs treated in the presence or absence (+/−) of an antibody to the extracellular domain of LINGO1; or in the presence of T3+PDGF in the presence or absence of the antibody to LINGO1. CNPase 1 is a marker of myelination competence.

FIG. 2 shows a representative western blot analysis of rat GRP expression of myelin basic protein (MBP) in rat GRPs treated in the presence or absence (+/−) of an antibody to the extracellular domain of LINGO1; or in the presence of T3+PDGF in the presence or absence of the antibody to LINGO1.

FIG. 3 shows a representative fluorescent microscopic image (488 nm filter) of GRPs transduced with an Integrin β1-IRES-GFP lentivirus (left panel), which demonstrates GFP expression in the successfully transduced cells. The panel on the right is the same image taken using phase contrast optics.

FIG. 4 shows an illustrative FACS analysis of GRPs transduced with Integrin β1-IRES-GFP and Integrin α4-IRES-luciferase lentiviruses. The panel on the left plotting forward versus side scatter shows a distinct population of healthy GRPs. These cells are gated for GFP fluorescence analysis, as shown in the panel on the right. FL1 on the X-axis represents the signal generated by IRES-GFP.

FIG. 5 shows an illustrative FACS analysis of GRPs transduced with Integrin α4-IRES-luciferase, β1-IRES-GFP, and CXCR3 lentiviruses. The panel on the left shows the same distinct population of healthy GRPs shown in FIG. 4. These cells are gated for analysis, shown in the panel on the right. While a similar proportion of cells generate an FL1 signal corresponding to IRES-GFP compared to controls, these cells were labelled with anti-CXCR3 antibody conjugated to PE-Cy5 captured by the FL3 signal. A small, but significant 0.23% percent of gated cells express CXCR3.

FIG. 6 shows a series of illustrative MRI images from rats intra-arterially administered either GRPs genetically modified to express VLA4 and labeled with ferridex; or saline. The left panel shows a series of three MRI images, showing detection of GRPs in the brain (ipsilateral and contralateral to intra-arterial injection site). The right panel shows images taken from rats injected with saline (top) or ferridex-labeled GRPs. GRPs are apparent as dark spots throughout the brain.

### DETAILED DESCRIPTION OF THE INVENTION

The appended claims particularly point out features set forth herein. A better understanding of the features and advantages of the present disclosure will be obtained by reference to the following detailed description that sets forth illustrative embodiments, in which the principles described herein are utilized.

Several barriers exist to the development of effective cell-based therapies into the nervous system, e.g., the CNS. For example, where stem cells or stem-cell derived cells are transplanted into a subject, these cells may fail to differentiate into the desired cell type. Generally, delivery of cells into the target tissue for therapy utilizes surgical delivery through the target tissue thereby resulting in damage to tissue surrounding the injection site. Further, delivery of the cells is limited to a volume proximal to the injection site, which limits the therapeutic effect of the cells to a relatively small region of tissue. Yet another problem is that heterologous transplanted cells may by killed by the immune system (immune rejection).

Accordingly, provided herein are cell-based compositions suitable for transplantation and methods for their use.

### I. Therapeutic Cells and Related Compositions

### Types of Therapeutic Cells

A variety of therapeutic cells and cell types are optionally used in the methods described herein. In some embodiments, the therapeutic cells are multipotent stem cells, e.g., multipotent neuroepithelial stem cells, which are capable of giving rise to a limited number of different cell lineages such as neuronal and glial cell lineages. In other embodiments, the therapeutic cells are lineage-restricted stem cells, e.g., neural-restricted precursors (NRPs) or glial-restricted precursors (GRPs). In other embodiments, therapeutic cells include partially or fully differentiated cells, e.g., oligodendrocytes, Schwann cells, astroglia, dopaminergic neurons, cholinergic neurons, GABAergic neurons, or glutamatergic neurons. In some embodiments, therapeutic cells include a mixed population of therapeutic cells, e.g., NRPs and GRPs; GRPs and oligodendrocytes; GRPs and neurons; NRPs and oligodendrocytes, etc. The selection of therapeutic cells will depend on a number of factors including the particular condition to be treated and the primary cell type affected by the condition. For example, in some embodiments where a condition is characterized by demyelination, therapeutic cells that include GRPs capable of differentiating into oligodendrocytes (e.g., myelination-competent oligodendrocytes) are administered to restore myelin in an afflicted subject. In other embodiments, where a condition is characterized by extensive neuronal loss, therapeutic cells that include, e.g., NRPs are used to replace neurons that were damaged or destroyed in the afflicted subject. In some embodiments, therapeutic cells are obtained directly from a primary tissue source such as an embryonic, fetal, or adult tissue, and followed by expansion and/or differentiation ex vivo. In other embodiments, the therapeutic cells are obtained by passaging established stem cell lines. In some embodiments, therapeutic cells are obtained by differentiating pluripotent stem cells such as embryonic stem (ES) cells or induced pluripotent stem (iPS) cells (as described in, e.g., Park et al (2008), Nat Protoc, 3(7): 1180-1186). In some embodiments, the therapeutic cells to be administered to a subject are heterologous to the subject. In other embodiments, the therapeutic cells to be administered are autologous, which reduces or eliminates the potential for immune rejection of the therapeutic cells after transplantation.

### Growth and Differentiation of Therapeutic Cells

In some embodiments, the therapeutic cells used as described herein are lineage-restricted progenitor cells, e.g., glial-restricted progenitors (GRPs), neural-restricted progenitors (NRPs), and motor neuron progenitors. In some embodiments the progenitor cells are motor neuron progenitors. In some embodiments, the progenitor cells are GRPs. Isolation of GRPs and their culture is described in, e.g., Herrera et al (2001), Exp Neurol, 171(1): 11-21, Rao et al (1998) Proc Natl Acad Sci USA, 95(7):3996-4001. See also, U.S. Pat. Nos. 6,235,527 and 6,900,054.

In an exemplary embodiment, GRPs are isolated and cultured as follows. Trunk segments including the last 10 somites are dissected from E13.5 rat embryos and triturated to remove the neural tube from the somites. The neural tube is then dissociated by digestion with 0.05% trypsin at 37° C. to obtain a cell suspension. E-NCAM-positive cells, previously shown to be NRPs (Mayer-Proschel et al, (1997) Neuron, 19, 773-785), are removed by plating the suspension onto E-NCAM-coated dishes for 20 minutes at 37° C. The supernatant is then collected and A2B5⁺ GRP cells are isolated by positive immunopanning. Cells were plated onto A2B5 antibody-coated dishes for 20 minutes at 37° C. The supernatant is then removed, the plate washed with culture medium, and the bound A2B5⁺ cells are collected by scraping them off the plate. Cells are placed into a 75 cm² tissue culture flask, coated with fibronectin/laminin solution (FN/LN) containing DMEM-F-12-BS with 10 ng/ml of bFGF (10 ml total for a 175 cm² flask), and grown in an incubator at 37° C., in 6-7.5% C0₂. After 5-7 days, when cultures reach 50-70% confluence, cells are harvested for transplantation. This procedure yields 98% A2B5⁺ cells as confirmed by immunostaining of a control aliquot of such cultures with GalC, GFAP, and A2B5 antibodies before transplantation. Previous clonal studies have confirmed that these A2B5⁺ cells are all GRP cells and can differentiate into oligodendrocytes and astrocytes but not neurons (Mayer-Proschel et al., 1997; Rao et al., 1998).

In some embodiments, GRPs are treated with an agent that induces differentiation of GRPs into oligodendrocytes (e.g., myelination-competent oligodendrocytes). Characteristic features of myelination-competent oligodendrocytes include, but are not limited to, expression of myelin basic protein (MBP) and CNPase1.

In some embodiments, differentiation of GRPs into myelination-competent oligodendrocytes includes removing FGF2 from the culture medium and adding Platelet-derived growth factor (PDGF; 20 ng/ml) and triiodothyronine (T3; 30-200 ng/ml) for 4 days. In other embodiments, GRP differentiation into myelination-competent oligodendrocytes includes treatment with a LINGO antagonist, as described herein, but not PDGF or T3 treatment. In other embodiments, GRP differentiation includes treating GRPs with one or both of T3 and PDGF. In some embodiments, GRPs are differentiated into myelination competent oligodendrocytes by treatment of the GRPs with an inhibitor of RhoA, e.g., the C3-05 protein as described in Dubreuil et al (2003), J Cell Biol, 162(2):233-243, siRNA to Rho A (as described in, e.g., Hengst et al (2006), J Neurosci, 26(21):5727-5732), or a dominant negative Rho A (as described in, e.g., Liang et al (2004), J. Neurosci., 24(32):7140-7149), or a Rho kinase inhibitor, e.g., Fasudil.

In some embodiments, GRPs are induced to become myelination-competent, prior to administration to a subject, by contacting the GRPs with a LINGO-blocking agent, e.g., an agent that inhibits expression of LINGO LINGO activity, e.g., LINGO homotypic interactions. Examples of LINGO-blocking agents include, but are not limited to, LINGO1-Fc fusion proteins, antibodies against the extracellular domain of LINGO 1, LINGO1-dominant negative proteins, small molecules that inhibit LINGO homotypic interactions, LINGO1 RNAi, and LINGO1 antisense nucleic acids. Examples of LINGO blocking agents is described in, e.g., U.S. patent application Ser. No. 10/553,685 and WO publication no. WO/2006/002437, and Mi et al (2005), Nat Neurosci, 8(6):745-751.

In an exemplary embodiment, A2B5⁺ GRPs are plated on a substrate coated with poly-L-lysine and laminin (coating solution contained 15 μg/ml of each) in PDGF-free growth medium supplemented with 10 ng/ml CNTF and 15 nM triiodothyronine and are immediately treated with a LINGO blocking agent for about 3 days to about 4 weeks, e.g., about 4 days, 5 days, 1 week, 10 days, 2 weeks, 3 weeks, or another period from about 3 days to about 4 weeks. In some embodiments, where the LINGO blocking agent is a substantially purified LINGO1-Fc fusion protein, the concentration of LINGO1-Fc is about 5 μg/ml to about 50 μg/ml, e.g. about 7 μg/ml, 8 μg/ml, 10 μg/ml, 12 μg/ml, 15 μg/ml, 20 μg/ml, 25 μg/ml, 35 μg/ml, 40, or another concentration of substantially purified LINGO1-Fc fusion protein from about 5 μg/ml to about 50 μg/ml. The term “substantially purified,” as ised herein, refers to a component of interest which is at least 85% pure, at least 90% pure, at least 95% pure, at lease 99% pure or greater pure. In other embodiments, where the LINGO blocking agent is an antibody to the extracellular domain of LINGO1, the concentration of LINGO1 antibody is about 5 μg/ml to about 50 μg/ml, e.g. about 7 μg/ml, 8 μg/ml, 10 μg/ml, 12 μg/ml, 15 μg/ml, 20 μg/ml, 25 μg/ml, 35 μg/ml, 40, or another concentration of substantially purified LINGO-Fc fusion protein from about 5 μg/ml to about 50 μg/ml. In some embodiments, where the LINGO blocking agent is a LINGO1 siRNA, the concentration of LINGO1 siRNA is about 10 nM to about 100 nM, e.g., about 12 nM, 15 nM, 20, nM, 30 nM, 40 nM, 50 nM, 70 nM, or another concentration from about 10 nM to about 100 nM. LINGO1 siRNA is available from commercial sources, e.g., MISSION® siRNA (Catalog ID: SASI_Mm01_{—}00137609) from SIGMA-ALDRICH or Santa Cruz Biotechnology (catalog: sc-60938).

In some embodiments, the therapeutic cells to be administered are obtained by differentiating human embryonic stem cells (see, e.g., Bongso et al (2005), Stem Cell Rev, 1(2):87-98; or other pluripotent stem cells, e.g., induced pluripotent stem (iPS) cells (see, e.g., Yamanaka et al (2007), Cell Stem Cell, 1(1):39-49).

In some embodiments, oligodendrocytes are generated from human pluripotent cells (e.g. ES cells).

Differentiation of the pluripotent stem cells into oligodendrocytes may be accomplished by known methods for differentiating ES cells or neural stem cells into oligodendrocytes. For example, oligodendrocytes are generated by co-culturing pluripotent stem cells or neural stem cells with stromal cells, e.g., Hermann et al. (2004), J Cell Sci. 117(Pt 19):4411-22. In another example, oligodendrocytes are generated by culturing the pluripotent stem cells or neural stem cells in the presence of a fusion protein, in which the Interleukin (IL)-6 receptor, or derivative, is linked to the IL-6 cyotkine, or derivative thereof. Oligodendrocytes are optionally generated from the pluripotent stem cells by other methods, see, e.g. Kang et al., (2007) Stem Cells 25, 419-424; and Shin et al (2007), Stem Cells Dev, February; 16(1):131-41.

Any known method of generating neural stem cells from ES cells is optionally used to generate neural stem cells from pluripotent stem cells, See, e.g., Reubinoff et al., (2001), Nat, Biotechnol., 19(12):1134-40. For example, neural stem cells are generated by culturing the pluripotent stem cells as floating aggregates in the presence of noggin, or other bone morphogenetic protein antagonist, see e.g., Itsykson et al., (2005), Mol, Cell Neurosci., 30(1):24-36. In another example, neural stem cells are generated by culturing the pluripotent stem cells in suspension to form aggregates in the presence of growth factors, e.g., FGF-2, Zhang et al., (2001), Nat. Biotech., (19):1129-1133. In some cases, the aggregates are cultured in serum-free medium containing FGF-2. In another example, the pluripotent stem cells are co-cultured with a mouse stromal cell line, e.g., PA6 in the presence of serum-free medium comprising FGF-2. In yet another example, the pluripotent stem cells are directly transferred to serum-free medium containing FGF-2 to directly induce differentiation.

Neural stem cells derived from the pluripotent stem cells are optionally differentiated into neurons, oligodendrocytes, or astrocytes. Often, the conditions used to generate neural stem cells is also used to generate neurons, oligodendrocytes, or astrocytes.

Dopaminergic neurons play a central role in Parkinson's Disease and other neurodegenerative diseases and are thus of particular interest. In order to promote differentiation into dopaminergic neurons, pluripotent stem cells are optionally co-cultured with a PA6 mouse stromal cell line under serum-free conditions, see, e.g., Kawasaki et al., (2000) Neuron, 28(1):31-40. Other methods have also been described, see, e.g., Pomp et al., (2005), Stem Cells 23(7):923-30; U.S. Pat. No. 6,395,546, e.g., Lee et al., (2000), Nature Biotechnol., 18:675-679.

### Modifications of Cells

In some embodiments, the therapeutic cells described herein are modified (e.g., genetically modified or modified with a cross-linking agent) to facilitate their delivery to target sites in the CNS. In some embodiments, modifying the therapeutic cells includes modifying the cells to permit their transport across the blood-brain barrier. Such modifications include, but are not limited to, expression of an exogenous cell surface protein on the therapeutic cells (“targeting ligand polypeptide”) that binds to a cell surface protein expressed on the blood brain barrier (BBB) (“targeting ligand receptor”), e.g., vascular cell adhesion molecule-1 (VCAM-1), insulin receptor, transferrin receptor, leptin receptor, lipoprotein receptor, and the IGF receptor. In some embodiments, the therapeutic cells are modified to express one or more exogenous targeting ligand polypeptides that interact with VCAM-1. In some embodiments, the exogenous targeting ligand polypeptide comprises an amino acid sequence that is at least 75% identical (e.g., 80%, 85%, 88%, 90%, 92%, 98%), or any other percent identical from 75% to 100% identical to the amino acid sequence of any of human, canine, or rodent Integrin α4, Integrin α9, Integrin αD, Integrin β1, Integrin β7, Ezrin, Moesin, LFA-1α (CD11a), CD18, VCAM-1, and Cathepsin G; or (ii) a heavy chain or light chain of antibody that binds specifically to human, rodent, or canine VCAM-1. In one embodiment, the exogenous targeting ligand polypeptide is a VLA-4 polypeptide or a variant thereof that interacts with VCAM-1. In some embodiments, the VLA-4 polypeptide is a heterodimer comprising (i) an Integrin α4 that is at least 75% identical (e.g., 80%, 85%, 88%, 90%, 92%, 98%), or any other percent identical from 75% to 100% identical to the amino acid sequence of any of human, canine, or rodent Integrin α4; and (ii) an Integrin β1 that is at least 75% identical (e.g., 80%, 85%, 88%, 90%, 92%, 98%), or any other percent identical from 75% to 100% identical to the amino acid sequence of any of human, canine, or rodent Integrin β1. In some embodiments, the targeting ligand polypeptide that interacts with VCAM-1 (i) comprises an amino acid sequence shorter than the full length amino acid sequence of any of human, canine, or rodent Integrin α4, Integrin α9, Integrin αD, Integrin β1, Integrin β7, Ezrin, Moesin, VCAM-1, and Cathepsin G; and (ii) comprises the extracellular domain (ECD) of any of the foregoing polypeptides.

In some embodiments, the therapeutic cells are modified to express an exogenous chemokine receptor polypeptide, e.g., a CXCR or a CCR. In some embodiments, the expression of an exogenous chemokine receptor on the therapeutic cells described herein facilitates the migration of therapeutic cells to sites where they will provide the greatest therapeutic benefit, e.g., sites of inflammation in the CNS, which occur in a number of CNS pathologies e.g., multiple sclerosis and Alzheimer's disease. In some embodiments, the exogenous chemokine receptor to be expressed is a CXC chemokine receptor, e.g., a polypeptide comprising an amino acid sequence that is at least 75% identical (e.g., 80%, 85%, 88%, 90%, 92%, 98%), or any other percent identical from 75% to 100% identical to the amino acid sequence of any of human, canine, or rodent CXCR1, CXCR2, CXCR3, CXCR4, CXCR5, CXCR6, or CXCR7. In other embodiments, the exogenous chemokine receptor to be expressed is a CCR chemokine receptor, e.g., a polypeptide comprising an amino acid sequence that is at least 75% identical (e.g., 80%, 85%, 88%, 90%, 92%, 98%), or any other percent identical from 75% to 100% identical to the amino acid sequence of any of human, canine, or rodent CCR1, CCR2, CCR3, CCR4, CCR5, CCR6, CCR7, CCR8, CCR9, CCR10, or CCR11.

In some embodiments, therapeutic cells are modified to express a targeting ligand polypeptide on their cell surface by introducing into the target cells one or more exogenous nucleic acids (e.g., expression vectors) encoding one or more targeting ligand polypeptides and configured to permit expression of the one or more polypeptides, e.g., a targeting ligand polypeptide, a chemokine receptor, or a reporter polypeptide. In some embodiments, a single expression vector encoding two separate expression cassettes is used to drive expression of two or more polypeptides of interest. In other embodiments, multiple (e.g., two or more) expression vectors are introduced into the therapeutic cell to drive expression of two or more polypeptides of interest.

In some embodiments, the therapeutic cells to be administered comprise one or more exogenous nucleic acids encoding a targeting ligand polypeptide that binds to a targeting ligand receptor found on the BBB. Examples of such nucleic acids include, but are not limited to those that hybridize under high stringency conditions with a nucleic acid encoding human, canine, or rodent Integrin α4, Integrin α9, Integrin αD, Integrin β1, Integrin β7, Ezrin, Moesin, VCAM-1, Cathepsin G, a heavy chain immunoglobulin directed to VCAM-1, or a light chain immunoglobulin directed to VCAM-1.

In some embodiments, the therapeutic cells to be administered are immortalized by introducing one or more expression vectors (e.g., a recombinant virus) encoding an immortalizing protein, e.g., V-myc, c-Myc, and SV40-T antigen. In some embodiments, the therapeutic cellss are conditionally immortalized by introducing one or more expression vectors encoding an immortalizing protein with an activity that is conditionally controlled by an exogenous agent (e.g., Myc-ER induced by RU486). In one embodiment, GRPs are immortalized by transduction with a lentivirus expression vector for expression of V-myc or SV40-T-antigen.

Generally, the exogenous nucleic acids introduced into the therapeutic cells described hererein will include expression control elements, such as promoters, enhancers, poly-adenylation signals. In some embodiments, a promoter will be a constitutive promoter, e.g., a CMV promoter, EFα promoter, or an SV40 promoter. In other embodiments, the promoter is a regulatable promoter, e.g., an inducible or repressible promoter. Regulatable promoters include, but are not limited to, tet-responsive promoters induced (“Tet-On”) or repressed (“Tet-Off”) by tetracycline and doxycycline. See, e.g., Dhawan et al., Somat. Cell. Mol. Genet., 21: 233 (1995); Gossen et al., Science, 268: 1766 (1995); Gossen et al., Science, 89: 5547 (1992); Shockett et al., Proc. Natl. Acad. Sci. USA, 92, 6522 (1995)), hypoxia-inducible nuclear factors (Semenza et al., Proc. Natl. Acad. Sci. USA, 88, 5680 (1991); Semenza et al., J. Biol. Chem., 269, 23757)), steroid-inducible elements and promoters, such as the glucocorticoid response element (GRE) (Mader and White, Proc. Natl. Acad. Sci. USA, 90, 5603 (1993)), and the fusion consensus element for RU486 induction (Wang et al., Proc. Natl. Acad. Sci. USA, 91:818 (1994)). In some embodiment, the promoter is cell type-specific, e.g., a myelin basic protein (MBP) promoter (for oligodendrocyte-specific expression) or a neuron-specific enolase (NSE) for neuron-specific expression.

In some embodiments, vectors include markers. In some embodiments, markers are selectable markers, which can be positive, negative, or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see e.g., WO 92/08796; and WO 94/28143). A large variety of such vectors are generally available.

Delivery of exogenous nucleic acids to a therapeutic cell is accomplished by any means, e.g., transduction, e.g., using recombinant viruses; transfection, with naked DNA, e.g., an expression vector for a polypeptide of interest, liposomes, association with polycations, calcium phosphate-mediated transformation, electroporation. A number of transfection techniques are described in, e.g., Graham et al., Virology, 52, 456 (1973), Sambrook et al., Molecular Cloning: A Laboratory Manual, Cold Spring Harbor Laboratories, New York (1989), Davis et al., Basic Methods in Molecular Biology, Elsevier (1986) and Chu et al., Gene, 13, 197 (1981). Particularly suitable transfection methods include calcium phosphate co-precipitation (Graham et al., Virol., 52, 456 (1973)), electroporation (Shigekawa et al., BioTechniques, 6, 742 (1988)), liposome-mediated gene transfer (Mannino et al., BioTechniques, 6, 682 (1988)), and lipid-mediated transfection (Felgner et al., Proc. Natl. Acad. Sci. USA, 84, 7413 (1987)).

In some embodiments, expression vectors are used to deliver exogenous nucleic acids to therapeutic cells. Vectors include, but are not limited to, isolated nucleic acid, e.g., plasmid-based vectors which, in some cases, are extrachromosomally maintained and viral vectors, e.g., recombinant adenovirus, retrovirus, lentivirus, herpesvirus, including cytomegalovirus, poxvirus, papilloma virus, or adeno-associated virus (AAV), including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Exemplary viral vectors are described below.

### Retroviral Vectors

Retroviral vectors exhibit several distinctive features including their ability to stably and precisely integrate into the host genome providing long-term transgene expression. These vectors are optionally manipulated ex vivo to eliminate infectious gene particles to minimize the risk of systemic infection and patient-to-patient transmission. In certain embodiments, the pseudotyped retroviral vectors alter host cell tropism.

### Lentiviruses

Lentiviruses are derived from a family of retroviruses that include human immunodeficiency virus and feline immunodeficiency virus. However, unlike retroviruses that only infect dividing cells, lentiviruses can infect both dividing and nondividing cells. For instance, lentiviral vectors based on human immunodeficiency virus genome are capable of efficient transduction of cardiac myocytes in vivo. Although lentiviruses have specific tropisms, pseudotyping the viral envelope with vesicular stomatitis virus yields virus with a broader range (Schnepp et al., Meth. Mol. Med., 69:427 (2002)).

### Adenoviral Vectors

Adenoviral vectors are optionally rendered replication-incompetent by deleting the early (E1A and E1B) genes responsible for viral gene expression from the genome and are stably maintained into the host cells in an extrachromosomal form. These vectors have the ability to transfect both replicating and nonreplicating cells and, in particular, these vectors have been shown to efficiently infect cardiac myocytes in vivo, e.g., after direction injection or perfusion. Adenoviral vectors have been shown to result in transient expression of therapeutic genes in vivo, peaking at 7 days and lasting approximately 4 weeks. The duration of transgene expression is optionally improved in systems utilizing cardiac specific promoters. In addition, adenoviral vectors are optionally produced at very high titers, allowing efficient gene transfer with small volumes of virus.

### Adeno-Associated Virus Vectors

Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to infect replicating and nonreplicating cells and are believed to be nonpathogenic to humans.

In one embodiment, recombinant AAV (rAAV) is employed to deliver a transgene to therapeutic cells. Differentiation is induced by placing subconfluent therapeutic cells in DMEM containing 2% horse serum and standard concentrations of glutamine and penicillin-streptomycin for an interval of four days prior to transduction.

### Herpesvirus/Amplicon

Herpes simplex virus 1 (HSV-1) has a number of important characteristics that make it an important gene delivery vector in vivo. There are two types of HSV-1-based vectors: 1) those produced by inserting the exogenous genes into a backbone virus genome, and 2) HSV amplicon virions that are produced by inserting the exogenous gene into an amplicon plasmid that is subsequently replicated and then packaged into virion particles. HSV-1 can infect a wide variety of cells, both dividing and nondividing, but has strong tropism towards nerve cells. It has a very large genome size and can accommodate very large transgenes (>35 kb). Herpesvirus vectors are particularly useful for delivery of large genes.

Therapeutic cells modified by any of the methods described herein to facilitate transport across the BBB are suitable for the manufacture of a medicament to suitable for treatment of a nervous system disorder, e.g., a CNS nervous system disorder as described.

### II. Use of Therapeutic Cells

In some embodiments, the therapeutic cells described herein are provided to a subject (e.g., a human, a non-human primate, a dog, a rabbit, or a rodent) thereof by an intra-arterial route of administration, which results in greatly reduced hemodilution of the administered cells relative to, e.g., intravenous administration of therapeutic cells. Further, intra-arterial administration of therapeutic cells that have been modified to cross the BBB, as described herein, results in dispersal of the therapeutic cells throughout the brain, rather than only a single locus, e.g., as occurs after injection directly into the brain. For example, by intra-arterial administration, therapeutic cells are delivered to two or more brain regions that are separated by a distance of at least about 0.05% to about 50% of the width, length, or height of the subject's brain, e.g., at least about 0.06%, 0.08%, 0.1%, 0.2%, 0.5%, 2%, 4%, 6%, 10%, 15%, 18%, 20%, 25%, 30%, 35%, 40%, 47%, 48%, or another distance from about 0.05% to about 50% of the width, length, or height of the subject's brain. In some embodiments, depending on the dimensions of the subject's brain, the distance between the two or more brain regions is about 0.01 cm to about 5 cm, e.g., about 0.02 cm, 0.05 cm, 0.07 cm, 0.08 cm, 0.1 cm, 0.2 cm, 0.3 cm, 0.4 cm, 0.5 cm, 1.0 cm, 1.2 cm, 2.0 cm, 2.5 cm, 3.2 cm, 3.5 cm, 4.5 cm, or another distance from about 0.01 cm to about 5 cm.

In some embodiments, the number of therapeutic cells to be administered to a subject in is at least about 1×10⁵ to about 1×10⁸ cells, e.g., about 2×10⁵ cells, 3×10⁵ cells, 4×10⁵ cells, 7×10⁵ cells, 1×10⁶ cells, 2×10⁶ cells, 3×10⁶ cells, 4×10⁶ cells, 7×10⁶ cells, 8×10⁶ cells, 1×10⁷ cells, 2×10⁷ cells, 3×10⁷ cells, 4×10⁷ cells, 5×10⁷ cells, 6×10⁷ cells, 7×10⁷ cells, 8×10⁷ cells, or another number of cells from at least about 1×10⁵ to about 1×10⁸ cells.

In some embodiments, the cells are administered at a concentration of about 2×10³ cells/μl to about 5×10⁴ cells/μl of administered cell suspension solution. The cells to be administered are administered in any sterile, physiologically acceptable isotonic solution, e.g., Hanks balanced salt solution solution, phosphate-buffered saline, citrate-buffered saline, or another physiologically compatible solution. In some embodiments, the pH and isotonicity of the suspension solution are adjusted (e.g., in pH and osmolarity) to obtain a cell suspension solution adapted for intra-arterial administration. In some embodiments, where the therapeutic cells have been genetically modified by introduction of an inducible expression vector, the cell suspension contains an appropriate induction agent, e.g., Doxycycline (in the case of a Tet-On promoter) at a concentration sufficient to induce expression of the inducible vector in the therapeutic cell.

In one embodiment, the cells are administered via a syringe or other device suitable for administration of cells into an artery under visual guidance by CT fluoroscopy. In one example, the device includes a needle for intra-arterial administration, a container in fluid communication with the needle, and within the container, a solution containing a plurality of transplantation-competent GRPs (e.g., GRPs that are genetically modified to cross the BBB when administered to a subject) and a pharmaceutically-acceptable carrier.

In some embodiments, while administering therapeutic cells to a subject by an administration device, e.g., a syringe, a small amount of arterial-blood from the subject is mixed ex vivo with therapeutic cells remaining in the administration device to form an ex vivo composition comprising arterial blood and a therapeutic cell. Such a composition is useful, e.g., for assessing a host immune response (e.g., a lymphocyte response) to the administered therapeutic cells. Methods and assays for determining, e.g., lymphocyte activation in response to an antigen include, e.g., Kruisbeek et al (2004), Curr Protoc Immunol, Chapter 3:Unit 3.12.

In some embodiments, the therapeutic cells are administered into, e.g., the carotid artery, femoral artery, intercostal arteries, or vertebral arteries. In some embodiments, where a subject is in need of therapeutic cells in the spinal cord, therapeutic cells are administered via intercostal arteries.

### Immunoprotection of Transplanted Cells

In some embodiments, following administration of the therapeutic cells into the circulation of a subject, the subject is administered an agent that inhibits the transport of immune cells across the BBB (“transport inhibitor”). In some embodiments, inhibitions of the ability of immune cells to translocate across the BBB will reduce or prevent immune rejection of the transplanted therapeutic cells, particularly those that have made it across the BBB into the CNS. In one embodiment, the transport inhibitor is an antibody against Integrin α4 (a subunit of VLA4), e.g., antibody known commercially as TYSABRI® (natalizumab) (Elan and Biogen-Idec). In other embodiments, the transport inhibitor is a small molecule inhibitor of Integrin α4, Integrin β1, or VLA4. See, e.g., Carpenter et al (2007), J Med Chem, 50(23):5863-5867. In some embodiments, the transport inhibitor is co-administered to the subject with an immunosuppressive drug. Examples of suitable immunosuppressive drugs include, but are not limited to, minocycline, tacrolimus, cyclosporin, rapamicin, methotrexate, cyclophosphamide, azathioprine, mercaptopurine, mycophenolate, or FTY720), glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non-steroidal anti-inflammatory drugs (e.g., salicylates, arylalkanoic acids, 2-arylpropionic acids, N-arylanthranilic acids, oxicams, coxibs, or sulphonanilides), Cox-2-specific inhibitors (e.g., valdecoxib, celecoxib, or rofecoxib), leflunomide, gold thioglucose, gold thiomalate, aurofin, sulfasalazine, hydroxychloroquinine, TNF-α binding proteins (e.g., infliximab, etanercept, or adalimumab), abatacept, anakinra, interferon-β, interferon-γ, interleukin-2, allergy vaccines, antihistamines, antileukotrienes, beta-agonists, theophylline, or anticholinergics.

### Tracking Therapeutic Cells In Vivo

After administration to a subject, therapeutic cells are optionally, but are not necessarily, detected and/or tracked within a region of the subject's nervous system (e.g., the subject's brain) by a noninvasive detection method. Examples of noninvasive detection methods include, but are not limited to, magnetic resonance imaging (MRI), single photon emission computed tomography (SPECT), X-ray computed tomography (CT), positron emission tomography (PET), fluorescence molecular tomography (FMT), and bioluminescence tomography (BLT). A number of methods are optionally used to facilitate noninvasive tracking of therapeutic cells. In some embodiments, therapeutic cells are labeled prior to administration with an agent suitable for detection in vivo by magnetic resonance-based detection methods (e.g., magnetic resonance imaging). Examples of such suitable agents include, but are not limited to, iron oxide particles, e.g., superparamagnetic iron oxide (SPIO) particles (e.g., Feridex®), as described in, e.g., Neri et al (2008), Stem Cells, 26:505-516; or, alternatively, liquid perfluorocarbon nanoparticles as described in Partlow et al (2007), The FASEB J, 21(8):1647-1654.

In one embodiment, therapeutic cells are genetically modified to express a reporter protein the amino acid sequence of which contains about 50 to about 250 lysines, e.g., 55 lysines, 70 lysines, 80 lysines, 100 lysines, 150 lysines, 200 lysines, 220 lysines, or another number of lysines from about 50 lysines to about 250 lysines, where at least 10 of the lysines are consecutive. The high lysine content of the just-mentioned reporter protein allows on chemical-exchange saturation transfer (CEST) imaging of the protein and cells expressing it. See, e.g., Ward et al (2000), J Magn Reson, 143:79-87 and Gilad et al (2007), Nat Biotechnol, 25(2):217-219. Subsequently, the labeled therapeutic cells are detected and/or imaged by magnetic resonance imaging (MRI) or magnetic resonance spectroscopy. SPIO particles or other labeling reagent are optionally introduced into therapeutic cells by a number of techniques including, but not limited to, lipofection or magnetoelectroporation (see, e.g., Walczak et al (2006), Nanomedicine 2(2):89-94.

In some embodiments, therapeutic cells are genetically modified to express an enzyme reporter protein. Examples of such enzymes include, but are not limited to, luciferase, β-galactosidase, Herpes thymidine kinase, and genetically modified versions thereof that retain at least 50% of the enzymatic activity. See, e.g., Shah et al (2008), 28(17):4406-4413; Jacobs et al (2001), Cancer Res. 2001, 61(7):2983-2995; and Louie (2006), Methods Mol Med, 124:401-417.

In some embodiments, therapeutic cells are genetically modified to express a reporter protein that emits green fluorescence, yellow fluorescence, or red fluorescence. Fluorescent proteins i include, e.g., Shaner et al (2007), J Cell Sci, 120(Pt 24):4247-4260; Shcherbo et al (2007), Nat Methods, 4(9):741-746.

In other embodiments, therapeutic cells are labeled, ex vivo, with a fluorescent probe that emits fluorescence in the near infrared range (e.g., a wavelength greater from than about 650 nm to about 1400 nm) thereby facilitating fluorescence detection or imaging of the therapeutic cells in vivo, e.g., by fluorescence molecular tomography (FMT). See, e.g., Rao et al (2007), Curr Opin Biotechnol, 18(1):17-25; Summer et al (2007), J Biomed Opt, 12(5):051504; and Swirksi et al (2007), 2(10):e1075. A number of NIR fluorescent probes are available commercially, e.g., from Invitrogen (Carlsbad, Calif.).

In some embodiments, expression of any of the above-mentioned reporter proteins is driven by a cell-type specific promoter, including, but not limited to, oligodendrocyte-specific promoters (e.g., a myelin basic protein (MPB) protein promoter, or a cyclic nucleotide phosphodiesterase CNPase promoter), and neuron-specific promoters (e.g., a synapsin promoter, a neuron-specific enolase promoter, or a CamKII promoter).

In some embodiments, following administration of therapeutic cells to a subject, the presence and location of the cells labeled by any of the above-mentioned methods is monitored at one or more time points (e.g., 2, 3, 4, 5, 6, 8, 10, 12, 15, 20, or 24 time points) following the administration. The therapeutic cells are optionally monitored at any time from about 6 hours to 1 year following administration of the cells, e.g., 12 hours, 24 hours, 2 days, 5 days, 1 week, 2 weeks, 3 weeks, 1 month, 2 months, 3 months, 4 months, 6 months, 10 months, or another time point from about 6 hours to about 1 year. In some embodiments, monitoring of the administered therapeutic cells in the subject is repeated at regular time intervals, e.g., weekly, monthly, or quarterly. In some embodiments, the presence of administered therapeutic cells is monitored, as described herein, in a CNS tissue (e.g., the brain or spinal cord). In other embodiments, the presence of administered therapeutic cells is monitored in the PNS. In some embodiments, monitoring the administered therapeutic cells includes monitoring the presence of the therapeutic cells in the bloodstream.

### Conditions to be Treated by Administration of Therapeutic Cells

The therapeutic cells described herein are used to treat any condition of the nervous system where cell replacement or supplementation has therapeutic value. Accordingly, in some embodiments, therapeutic cells are used to treat a demyelinating condition. In one embodiment, where the condition to be treated is a demyelinating condition, the therapeutic cells to be administered are myelination-competent therapeutic cells, e.g., GRPs, oligodendrocytes, O4⁺ pre-myelinating oligodendrocytes treated with a LINGO blocking agent as described herein. A demyelinating condition may occur in the CNS or the PNS. Examples of CNS demyelinating conditions that are treated by the methods described herein include, but are not limited to, multiple sclerosis, optic neuritis, acute transverse myelitis, acute disseminated encephalomyelitis, Devic's disease, and acute hemorrhagic leukoencephalitis, hereditary disorders (e.g., Phenylketonuria and other aminoacidurias, Tay-Sachs, Niemann-Pick disease, Gaucher's disease, Hurler's syndrome, Krabbe's disease and other leukodystrophies, Adrenoleukodystrophies, Adrenomyeloneuropathy, Leber's hereditary optic atrophy and related mitochondrial disorders); Hypoxia and Ischemia (e.g., carbon monoxide toxicity and other syndromes of delayed hypoxic cerebral demyelination and progressive subcortical ischemic demyelination); nutritional deficiencies (Central pontine myelinolysis (may also be caused by Na fluxes); Demyelination of the corpus callosum (Marchiafava-Bignami disease); virally-induced demyelination (e.g., progressive multifocal leukoencephalopathy, subacute sclerosing panencephalitis, and tropical spastic paraparesis/HTLV-1-associated myelopathy.

In the PNS, demyelinating conditions to be treated include, but are not limited to, Guillain-Barré syndrome, chronic inflammatory demyelinating polyneuropathy, diabetic neuropathy, and HIV-associated neuropathy.

In other embodiments, the condition to be treated is a neurodegentative condition or a neuropsychiatric condition. In one embodiment, where the condition to be treated is a neurodegenerative condition, the therapeutic cell to be administered are neurons, neural stem cells, or neural progenitor cells (e.g., motor neuron progenitors) as described herein. Examples of neurodegenerative conditions that are treated by the methods described herein include, but are not limited to, Alzheimer's Disease, Huntington's Disease, Parkinson's Disease, HIV-associated dementia, Amyotrophic Lateral Sclerosis, Multiple System Atrophy, degenerative retinal disease (e.g., macular degeneration), Schizophrenia, Pick's disease, Alexander disease, Alper's disease, Ataxia telangiectasia, Batten disease (also known as Spielmeyer-Vogt-Sjogren-Batten disease), Canavan disease, Cockayne syndrome, Corticobasal degeneration, Creutzfeldt-Jakob disease, Kennedy's disease, Krabbe disease, Lewy body dementia, Machado-Joseph disease (Spinocerebellar ataxia type 3), Neuroborreliosis, Pelizaeus-Merzbacher Disease, Primary lateral sclerosis, Prion diseases, Refsum's disease, Sandhoff disease, Schilder's disease, Spielmeyer-Vogt-Sjogren-Batten disease (also known as Batten disease), Spinocerebellar ataxia (multiple types with varying characteristics), Spinal muscular atrophy, Steele-Richardson-Olszewski disease, Tabes dorsalis, or any combination thereof.

In some embodiments, the methods described herein are used to treat acute neurodegenerative conditions, which include, but are not limited to stroke (e.g., thromboembolic stroke, focal ischemia, global ischemia, or transient ischemia), ischemia resulting from a surgical technique involving prolonged halt of blood flow to the brain, head trauma, spinal trauma, or any combination thereof.

In further embodiments, the methods described herein are used to treat a psychotic disorder. At least some psychotic orders are impacted by a decrease in neurogenesis (e.g., schizophrenia). See, e.g., Toro et al (2007). Thus, in some embodiments, a psychotic disorder is treated by the methods described herein. Examples of psychotic disorders include, but are not limited to, schizophrenia, schizoaffective disorder, schizophreniform disorder, brief psychotic disorder, delusional disorder, shared psychotic disorder (Folie a Deux), substance induced psychosis, and psychosis due to a general medical condition.

In some embodiments, the methods described herein are used to treat a subject suffering from a mood disorder. Examples of mood disorders include, but are not limited to, clinical depression, bipolar disorder, cyclothymia, and dysthymia.

In some embodiments, the methods described herein are used to treat a subject suffering from age-related cognitive decline.

Symptoms, diagnostic tests, and prognostic tests for each of the above-mentioned conditions are found, e.g., in the Diagnostic and Statistical Manual of Mental Disorders, 4th ed., 1994, Am. Psych. Assoc.; and Harrison's Principles of Internal Medicine©,” 16th ed., 2004, The McGraw-Hill Companies, Inc

For example, where the subject is at risk of or is suffering from multiple sclerosis, a set of standard criteria, such as the “McDonald Criteria” are optionally used for prognosis/diagnosis. See McDonald et al. (2001), Ann Neurol, 50(1): 121-127. Magnetic resonance imaging (MRI) of the brain and spine is optionally used to evaluate individuals with suspected multiple sclerosis. MRI shows areas of demyelination as bright lesions on T2-weighted images or FLAIR (fluid attenuated inversion recovery) sequences. Gadolinium contrast is used to demonstrate active plaques on T1-weighted images. Further, a prognostic biomarker assay of cerebrospinal fluid (CSF) obtained by lumbar puncture can provide evidence of chronic inflammation of the central nervous system. Specifically, CSF is tested for oligoclonal bands, which are immunoglobulins found in 85% to 95% of people with definite MS, albeit not exclusively in MS patients. Additional criteria for diagnosis of multiple sclerosis include, e.g., a reduction in visual evoked potentials and somatosensory evoked potentials, which are indicative of demyelination.

Where a neurodegenerative disorder affects a cognitive ability, a subject is optionally diagnosed by any one of a number of standardized cognitive assays, e.g., the Mini-Mental State Examination, the Blessed Information Memory Concentration assay, or the Functional Activity Questionnaire. See, e.g., Adelman et al. (2005), Am. Family Physician, 71(9):1745-1750. Indeed, in some cases a subject is diagnosed as having a high risk of developing a chronic neurodegenerative condition (e.g., Alzheimer's disease), even in the absence of overt symptoms. For example, the risk of Alzheimer's disease in a subject is determined by detecting a decrease in the volumes of the subject's hippocampus and amygdala, using magnetic resonance imaging. See, e.g., den Heijer et al. (2006), Arch Gen Psychiatry, 63(1):57-62. Assay of prognostic biomarkers in a sample from a subject are also useful in prognosis or diagnosis of a chronic neurodegenerative condition. For example, where the chronic neurodegenerative condition is Alzheimer's disease, prognostic biomarkers include, but are not limited to, total tau protein, phospho-tau protein, β-amyloid₁₋₄₂ peptide, β-amyloidi₁₋₄₀ peptide, complement component 1, q subcomponent (C1q) protein, interleukin 6 (IL-6) protein, apolipoprotein E (APOE) protein, α-1-antichymotrypsin protein, oxysterol (e.g., 24S-hydroxycholesterol), isoprostane (e.g., an F2-isoprostane), 3-nitrotyrosine, homocysteine, or cholesterol, or any combination thereof, e.g., the ratio of β-amyloid₁₋₄₂ peptide to β-amyloid₁₋₄₀ peptide.

The type of biological sample utilized in prognostic Alzheimer's biomarker assays will vary depending on the prognostic biomarker to be measured. Further, the relationship between the level of a prognostic biomarker and Alzheimer's risk varies depending on the particular biomarker, as well as on the biological sample in which the level of the biomarker is determined. In other words, the level of the biomarker in a biological sample is either directly correlated or inversely correlated with the risk of Alzheimer's Disease, as summarized in Table 1.

TABLE 1
ALZHEIMER'S DISEASE PROGNOSTIC BIOMARKERS

| | Biological | Correlation to | |
|---|---|---|---|
| Biomarker | Sample Type | Dementia Risk | Reference |
| tau protein | cerebrospinal | increased | Hampel et al. (2004), Mol Psychiatry, 9: 705-710 |
| | fluid (CSF) | | |
| phospho-tau protein | CSF | increased | Hampel et al. (2004), Arch Gen Psychiatry, 61: 95-102 |
| | | | Hansson et al. (2006), Lancet Neurol, 5(3): 228-234 |
| β-amyloid₁₋₄₂ peptide | CSF | decreased | Hampel et al. (2004), Mol Psychiatry, 9: 705-710 |
| Ratio of β-amyloid₁₋₄₂ | plasma | decreased | Graff-Radford et al. (2007), Arch Neurol, |
| peptide to β-amyloid₁₋₄₀ | | | 64(3): 354-362; |
| peptide | CSF | decreased | Hansson et al. (2007), Dement Geriatr Cogn |
| | | | Disord, 23(5): 316-20 |
| C1q protein | CSF | decreased | Smyth et al. (1994), Neurobiol Aging, 15(5): 609-614 |
| IL-6 protein | plasma | increased | Licastro et al. (2000), J Neuroimmunol, 103: 97-102; |
| | CSF | increased | Sun et al. (2003), Dement Geriatr Cogn Disord, |
| | | | 16(3): 136-44 |
| APOE protein | CSF | increased | Fukuyama et al. (2000), Eur Neurol, 43(3): 161-169 |
| α-1-antichymotrypsin | plasma | increased | Dik et al. (2005), Neurology, 64(8): 1371-1377. |
| protein | | | |
| oxysterol | CSF | increased | Papassotiropoulos et al. (2002), J Psychiatr Res, |
| | | | 36(1): 27-32 |
| isoprostane | CSF | increased | Montine et al. (2005), Antioxid Redox Signal, |
| | | | 7(1-2): 269-275 |
| 3-nitrotyrosine | CSF | increased | Tohgi et al. (1999), Neurosci Lett, 269(1): 52-54 |
| homocysteine | plasma | increased | Seshadri et al. (2002), N Engl J Med, 346(7): 476-83 |
| cholesterol | plasma | increased | Panza et al. (2006), Neurobiol Aging, |
| | | | 27(7): 933-940 |

### Combination Therapies

The therapeutic cell compositions described herein are optionally used in combination with other well known therapeutic reagents that are selected for their therapeutic value for the condition to be treated. In general, the compositions described herein and, in embodiments where combinational therapy is employed, other agents do not have to be administered with a cell-containing composition, and may, because of different physical and chemical characteristics, have to be administered by different routes. The initial administration is optionally made according to established protocols, and then, based upon the observed effects, the dosage, modes of administration and times of administration are optionally modified.

In certain instances, it is appropriate to administer a therapeutic cell composition described herein in combination with another therapeutic agent. By way of example only, if one of the side effects experienced by a patient upon receiving one of the PAK activator compositions described herein is nausea, then it is appropriate to administer an anti-nausea agent in combination with therapeutic cell administration. Or, by way of example only, the therapeutic effectiveness of one of the compounds described herein is enhanced by administration of an adjuvant (i.e., by itself the adjuvant may have minimal therapeutic benefit, but in combination with another therapeutic agent, the overall therapeutic benefit to the patient is enhanced). Or, by way of example only, the benefit experienced by a patient is increased by administering one of the compounds described herein with another therapeutic agent (which also includes a therapeutic regimen) that also has therapeutic benefit. In any case, regardless of the disease, disorder or condition being treated, the overall benefit experienced by the patient is either additive of the two therapeutic agents or the patient experiences a synergistic benefit.

The particular choice of secondary agents used will depend upon the diagnosis of the attending physicians and their judgment of the condition of the patient and the appropriate treatment protocol. The compounds are optionally administered concurrently (e.g., simultaneously, essentially simultaneously or within the same treatment protocol) or sequentially, depending upon the nature of the disease, disorder, or condition, the condition of the patient, and the actual choice of compounds used.

Therapeutically-effective dosages can vary when therapeutic agents are used in treatment combinations. For example, the use of metronomic dosing, i.e., providing more frequent, lower doses in order to minimize toxic side effects, is optionally used. Combination treatment further includes periodic treatments that start and stop at various times to assist with the clinical management of the patient.

For combination therapies described herein, dosages of the co-administered agents will, of course, vary depending on the type of co-drug employed, on the specific drug employed, on the disease or condition being treated and so forth. In addition, when co-administered with one or more biologically active agents, the therapeutic cell compositions provided herein are optionally administered either simultaneously with the biologically active agent(s), or sequentially. If administered sequentially, the attending physician will decide on the appropriate sequence of administering therapeutic cells in combination with the biologically active agent(s).

In any case, the multiple therapeutic agents (one of which is a therapeutic cell composition described herein) are optionally administered in any order, or even simultaneously. If simultaneously, the multiple therapeutic agents are optionally provided in a single, unified form, or in multiple forms. One of the therapeutic agents is optionally given in multiple doses, or both may be given as multiple doses. If not simultaneous, the timing between the multiple doses optionally vary from more than zero weeks to less than four weeks. In addition, the combination methods, compositions and formulations are not to be limited to the use of only two agents; the use of multiple therapeutic combinations are also envisioned.

The dosage regimen to treat, prevent, or ameliorate the condition(s) for which relief is sought, is optionally modified in accordance with a variety of factors. These factors include the disorder from which the subject suffers, as well as the age, weight, sex, diet, and medical condition of the subject.

Thus, under such circumstances the dosage regimen employed can vary widely and therefore can deviate from the dosage regimens set forth herein.

The therapeutic agents described herein and combination therapies are optionally administered before, during or after the occurrence of a disease or condition, and the timing of administering the composition containing therapeutic cells or an additional therapeutic agent has the potential to vary. Thus, for example, the therapeutic agents are used as a prophylactic and are administered continuously to subjects with a propensity to develop conditions or diseases in order to prevent the occurrence of the disease or condition. In some embodiments the therapeutic agents and compositions is administered to a subject during or as soon as possible after the onset of the symptoms. In some embodiments the administration of the therapeutic agents is initiated within the first 48 hours of the onset of the symptoms, preferably within the first 48 hours of the onset of the symptoms, more preferably within the first 6 hours of the onset of the symptoms, and most preferably within 3 hours of the onset of the symptoms.

Exemplary Therapeutic Agents for Use in Combination with Therapeutic Cell Therapy

### Agents for Treating Multiple Sclerosis

Where a subject is suffering from or at risk of suffering from multiple sclerosis, a therapeutic cell composition described herein is optionally used together with one or more of the following exemplary multiple sclerosis therapeutic agents in any combination: Interferon β-1a, Interferon β-1b, glatiramer acetate (Copaxone®), mitoxantrone (Novantrone®), low dose naltrexone, Natalizumab (Tysabri®), Sativex®, Aimspro (Goats Serum), Trimesta (Oral Estriol), Laquinimod, FTY720 (Fingolimod), MBP8298, NeuroVax®, Tovaxin®, Revimmune, CHR-1103, BHT-3009, BG-12, Cladribine, daclizumab (Zenapax) Rituximab (Rituxan), cyclophosphamide, Campath, Fampridine-SR, MN-166, Temsirolimus, or RPI-78M.

### Agents for Treating Dementia (e.g., Alzheimer's Disease or AIDS-Related Dementia)

Where a subject is suffering from or at risk of suffering from dementia, a therapeutic cell composition described herein is optionally used together with one or more agents or methods for treating dementia in any combination. Examples of therapeutic agents/treatments for treating dementia include, but are not limited to any of the following: Flurizan™ (MPC-7869, r flurbiprofen), memantine, galantamine, rivastigmine, donezipil, tacrine, Aβ₁₋₄₂ immunotherapy, resveratrol, (−)-epigallocatechin-3-gallate, statins, vitamin C, or vitamin E.

### Agents for Treating Parkinson's Disease

Where a subject is suffering from or at risk of suffering from Parkinson's Disease, a therapeutic cell composition described herein is optionally used together with one or more agents or methods for treating Parkinson's disease in any combination. Examples of therapeutic agents/treatments for treating Parkinson's Disease include, but are not limited to any of the following: L-dopa, carbidopa, benserazide, tolcapone, entacapone, bromocriptine, pergolide, pramipexole, ropinirole, cabergoline, apomorphine, lisuride, selegiline, or rasagiline.

### Agents for Treating Amyotrophic Lateral Sclerosis

Where a subject is suffering from or at risk of suffering from Amyotrophic Lateral Sclerosis, a therapeutic cell composition described herein is optionally used together with one or more agents or methods for treating Amyotrophic Lateral Sclerosis in any combination. Examples of therapeutic agents/treatments for treating Parkinson's Disease include, but are not limited to any of the following: riluzole, insulin-like growth factor 1, or ketogenic diet.

### Agents for Treating Autoimmune Inflammatory, or Allergic Conditions

Where a subject is suffering from or at risk of suffering from an autoimmune, inflammatory disease, or allergic condition that affects the nervous system (see, e.g., Allan et al. (2003), Philos Trans R Soc Lond B Biol Sci, 358(1438): 1669-1677), a therapeutic cell composition described herein is optionally used together with one or more of the following therapeutic agents in any combination: immunosuppressants (e.g., tacrolimus, cyclosporin, rapamicin, methotrexate, cyclophosphamide, azathioprine, mercaptopurine, mycophenolate, or FTY720), glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non-steroidal anti-inflammatory drugs (e.g., salicylates, arylalkanoic acids, 2-arylpropionic acids, N-arylanthranilic acids, oxicams, coxibs, or sulphonanilides), Cox-2-specific inhibitors (e.g., valdecoxib, celecoxib, or rofecoxib), leflunomide, gold thioglucose, gold thiomalate, aurofin, sulfasalazine, hydroxychloroquinine, minocycline, TNF-α binding proteins (e.g., infliximab, etanercept, or adalimumab), abatacept, anakinra, interferon-β, interferon-γ, interleukin-2, allergy vaccines, antihistamines, antileukotrienes, beta-agonists, theophylline, or anticholinergics.

### Agents for Treating Thromboembolic Disorders

Where a subject is suffering from or at risk of suffering from a thromboembolic disorder (e.g., stroke), the subject is optionally treated with a therapeutic cell composition described herein in any combination with one or more other anti-thromboembolic agents. Examples of anti-thromboembolic agents include, but are not limited any of the following: thrombolytic agents (e.g., alteplase anistreplase, streptokinase, urokinase, or tissue plasminogen activator), heparin, tinzaparin, warfarin, dabigatran (e.g., dabigatran etexilate), factor Xa inhibitors (e.g., fondaparinux, draparinux, rivaroxaban, DX-9065a, otamixaban, LY517717, or YM150), ticlopidine, clopidogrel, CS-747 (prasugrel, LY640315), ximelagatran, or BIBR 1048.

### Anti-HIV Compounds

Where the subject is suffering from an HIV infection (e.g., suffering from AIDS), any of the therapeutic cell compositions described herein is optionally administered to the subject prophylactically or therapeutically to treat AIDs-related dementia in combination with one or more anti-HIV compounds administered to treat the HIV infection. Examples of anti-HIV compounds include, but are not limited to, AZT (zidovudine, Retrovir), ddI (didanosine, Videx), 3TC (lamivudine, Epivir), d4T (stavudine, Zerit), abacavir (Ziagen), and FTC (emtricitabine, Emtriva), tenofovir (Viread), efavirenz (Sustiva), nevirapine (Viramune), lopinavir/ritonavir (Kaletra), indinavir (Crixivan), ritonavir (Norvir), nelfinavir (Viracept), saquinavir hard gel capsules (Invirase), atazanavir (Reyataz), amprenavir (Agenerase), fosamprenavir (Telzir), tipranavir (Aptivus), or T20 (enfuvirtide, Fuzeon)

### EXAMPLES

The following specific examples are to be construed as merely illustrative, and not limitative of the remainder of the disclosure in any way whatsoever. Where reference is made to a URL or other such identifier or address, it is understood that such identifiers can change and particular information on the internet can come and go, but equivalent information can be found by searching the internet

### Example 1 Differentiation of GRPs into Myelination-Competent Oligodendrocytes

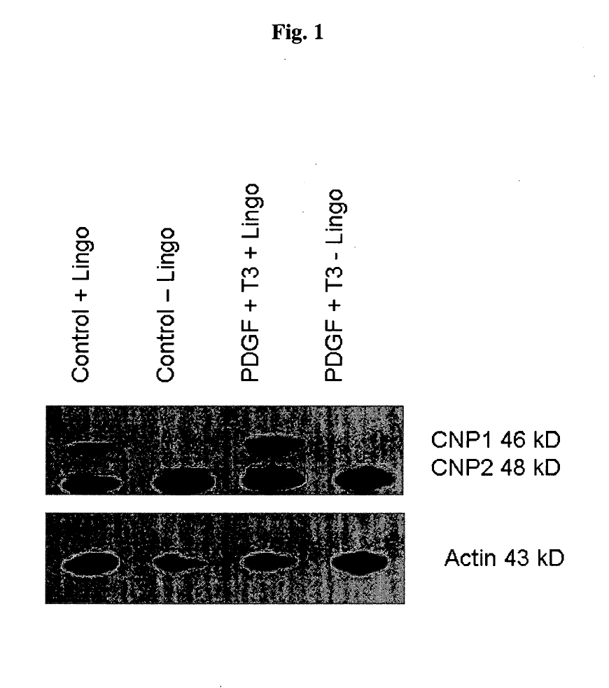

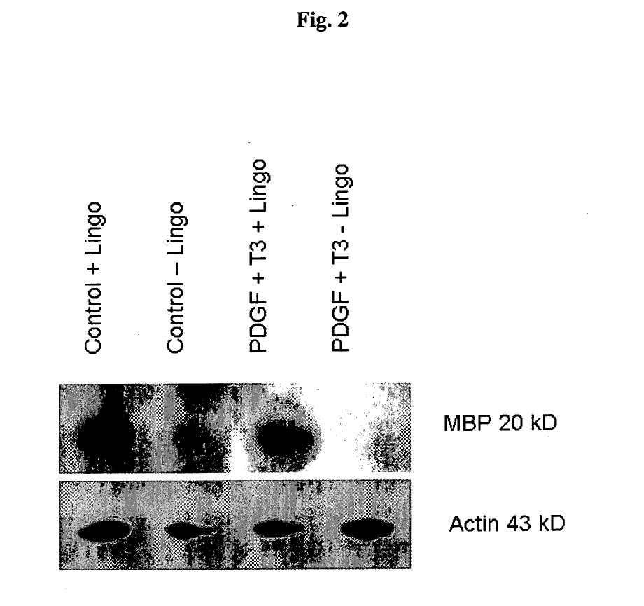

We sought to evaluate in vitro conditions for differentiating GRPs into myelination-competent oligodendrocytes. Accordingly, rat GRPs were plated on Poly-L-Lysine and laminin coated plates at 5×10⁵ cells per well of a six well dish in GRP media (described in Example 2). The following day, an antibody to the extracellular domain of LINGO1 with or without T3 plus PDGF, at a final concentration of 10 μg/ml (antibody), 30 ng/ml T3, 20 ng/ml PDGF. Medium containing these reagents were replaced daily. 72 hours after addition of the various reagent combinations, cell lysates were generated by lysing the cells with 200 ul of RIPA buffer and a cell scraper. Lysates were sonicated and protein concentration was determined by Bradford assay (Biorad). Lysate (25 μg) was added to each well of an SDS-PAGE gel, electrophoretically, and transferred to PVDF membrane (Immobilon). After blocking with 5% nonfat dry milk, immunodetection was carried out with rabbit anti-CNPAse (1:1000; CNP1) antibody (Chemicon) and anti-MBP antibody (Chemicon 1:500). As shown in FIG. 1, the myelination marker CNP1 was strongly induced by LINGO1 antibody treatment, and was enhanced by the addition of PDGF plus T3. As shown in FIG. 2, MBP expression followed a similar pattern. Based on these results, we concluded that GRPs are effectively differentiated by treatment with an antibody that binds to the extracellular domain of LINGO 1 with or without the addition of T3 plus PDGF.

### Example 2 Generation of Genetically Modified GRPs by Multiple Lentivirus Transduction

We sought to generate genetically modified GRPs that expressed VLA4 (a VCAM1 targeting ligand) on their surface.

### Methods

Rat glial restricted precursor (GRP) cells were isolated with modifications Rao et al (1998) 1998 Mar. 31; 95(7):3996-4001. The spinal cord was dissected from rats (E13-E13.5) with #5 forceps and plated in a Petri dish in DMEM/F12 media (Gibco, cat #11330). Spinal cords were incubated at 37° C. incubator in 10 mis of 0.005% Trypsin from bovine pancreas (Sigma # T1426) and 100 μl (10 mg/ml) stock solution DNASE-1 (Sigma cat #Dn25) for 10 minutes and then triturated. The cell suspension was then incubated for 10 minutes and triturated again. Five ml of GRP media was then added to the suspension, and the suspension was then centrifuged at 2000×g for 5 minutes. The resulting pellet was resuspended in 10 mis of GRP media to which 100 μl of DNAse-1 was then added. The suspension was then incubated at 37° C. for 10 minutes followed by gentle trituration and centrifugation at 1000 rpm for 5 minutes. The resulting cell pellet was then resuspended in 10 ml of GRP media and plated on a poly-L-lysine/laminin (15 μg/ml)-treated T25 flask. The cultures were maintained in a 5% CO₂ incubator at 37° C. Cultures were passaged when they reached 70-80% confluence. GRPs were immunopanned for enrichment, approximately two weeks after their isolation, based on their expression of the surface antigen A2B5 as described in Rao et al supra GRP medium

500 mis of GRP medium contained the following: 0.5 mg BSA, 10 mis of 50× stock B27; 5 mis of 100× stock N2; 5 mis of Pen/Streptomycin; 500 mis of DMEM/F12 media; 20 ng/ml of bFGF, 1 μg/ml of heparin. The GRP medium was sterilized by filtration through a 0.22 um filter.

### Immortalization of GRPs:

Flasks pre-coated with poly-L-lysine and laminin were necessary for adhesion. GRPs were maintained on poly-L-lysine/laminin coated flasks in serum free Ham's DMEM plus N2 and B27 supplements with 20 ng/μl of fibroblast growth factor (FGF) changed daily. V-Myc lentivirus and SV40-T antigen were used to immortalize rodent and human GRPs, respectively.

Lentiviral infection.

The following lentiviral constructs containing α4 and β1 integrins and chemokine receptors were obtained from Genecopoeia and packaged with VSV-G in 293T cells: α4-IRES-luciferase, β1-IRES-GFP, CXCR3, CXCR4, CCR3. The supernatant containing the virus was added to GRPs plated at 50-70% confluence and washed away after 24 hours. Cells were sorted by fluorescent activated cell sorting (FACS).

Fluorescent activated cell sorting (FACS). Evidence of expression of β1-IRES-GFP was obtained by visualization of GFP by fluorescence microscopy. GRPs were expanded from approximately 10⁵ cells to 10⁷ cells, washed with phosphate buffered saline (PBS), detached from the substrate with Sigma non-enzymatic cell dissociation media (cat C5789) and gently scraped before spinning down at 150×g for 5 minutes at room temperature. GRPs were labelled with 1:100 anti-CXCR3 antibody directly conjugated to PE-Cy5 for 15 minutes at 37 degrees, washed with PBS and resuspended in fresh PBS. Analysis and sorting was performed on a FACSVantage SE sorter on FL1 for GFP and FL3 for PE-CyS. Sorted cells were re-plated and expanded as described above.

### Results

### Transduction of Rat GRPs.

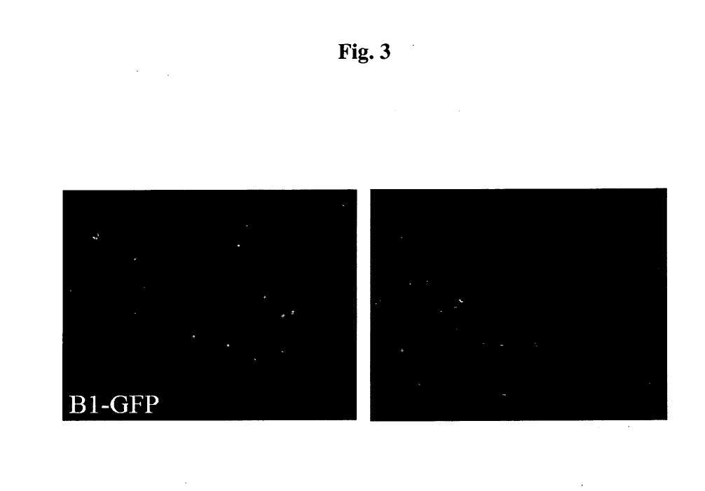

Rat GRPs were transduced with three recombinant lentiviruses containing α4-IRES-luciferase, β1-IRES-GFP, and, CXCR3. Expression of β1-IRES-GFP was screened by fluorescent microscopy using a 488 nm filter. As shown in FIG. 3, cells successfully transformed with the β1-IRES-GFP produced a green signal by fluorescence microscopy. In general, the GFP signal was poor during the growth phase of GRPs and more robust as the cells were 90-100% confluent as shown in the figure. The phase contrast imaging indicates that only a small percentage of cells were GFP positive.

FACS of GRPs Transduced with Multiple Lentivirus Vectors.

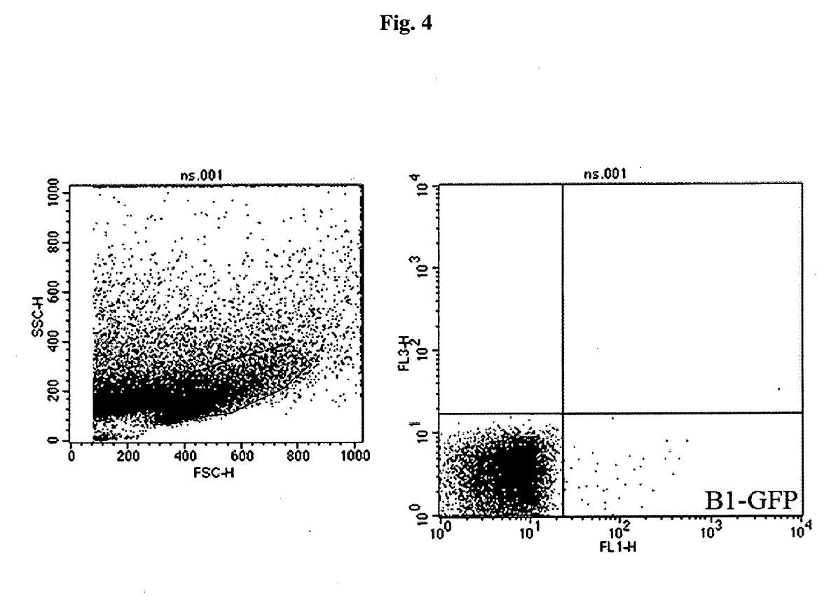

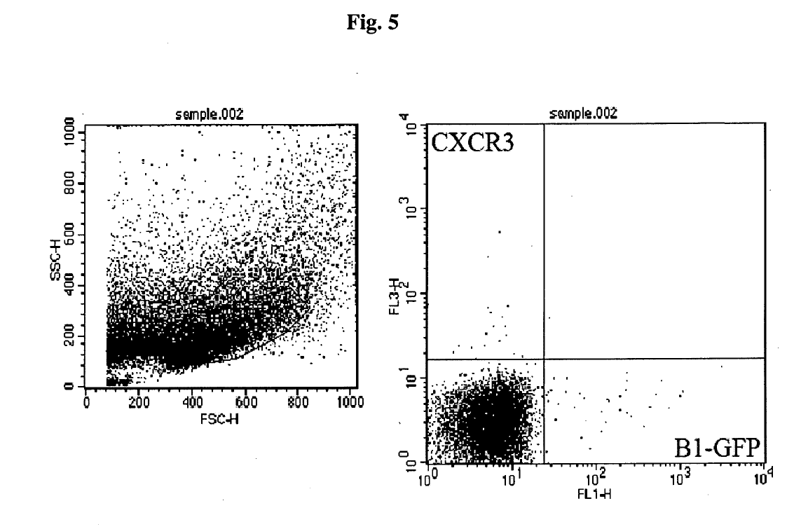

In order to demonstrate our ability to isolate a GRP line that expresses multiple constructs, approximately 10⁵ GRPs were transduced with α4-IRES-luciferase, β1-IRES-GFP, and, CXCR3 lentiviruses, expanded to about 10⁷, and then harvested for FACS. As shown in FIG. 4, side scatter versus forward scatter demonstrates a population of cells that represents healthy cells without debris. Approximately, 0.46% of this gated population of cells corresponded exhibited a positive FL1 signal generated by the expressed GFP. This proportion was consistent with the frequency of GFP expression we observed by fluorescence microscropy. We also stained the same cells for expression of CXCR3. When labelled with anti-CXCR3 antibody directly conjugated to PE-Cy5, this same population of GRPs reveal a positive FL3 signal in 0.23% of gated cells. Only a small population, 0.01% of gated cells were double positive for CXCR3 and GFP, as shown in FIG. 5. Cells identified as double positive by FACS analysis were then plated and expanded in culture by the methods described above.

### Example 3 Genetically Modified GRPs Administered by Intra-Arterial Administration are Delivered to the Brain

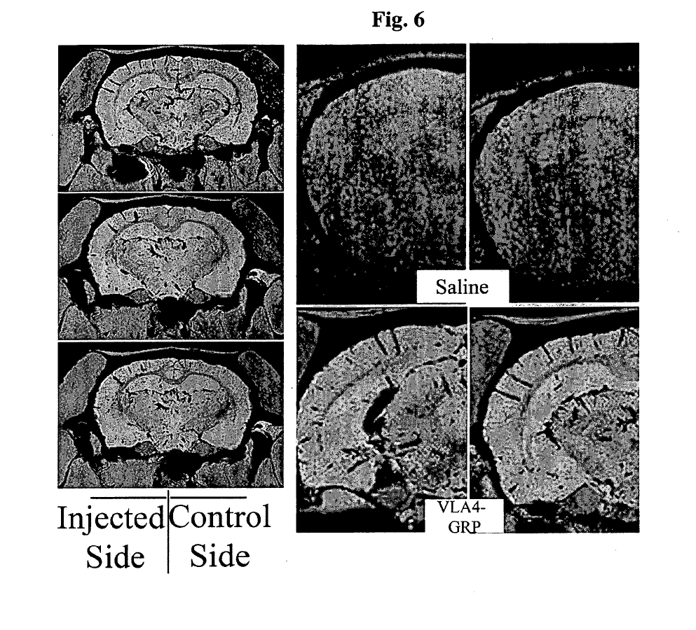

Naïve adult Lewis rats were given LPS 3 mg/kg 12 hours prior to transplantation. Lewis rat VLA4-expressing GRPs, generated by a method similar to that described in Example 2, were incubated with Ferridex at a dose of 25 μg/ml (Berlex Imaging, Wayne, N.Y., USA) that had been mixed with poly-L-lysine (375 ng/ml; Sigma-Aldrich, St. Louis, Mo., USA), incubated for 1 hr, and added to the cell culture for 24 hr. At the time of transplantation, 2 million GRPs in 500 μl of PBS were injected into the right common carotid artery after superficial dissection. Rats were then sacrificed prior to emergence from anesthesia and imaged with T2-star sequences on a 9.7T MRI. As shown in FIG. 6, GRPs are detected by ‘black’ signal throughout the brain, especially in a vascular distribution corresponding to deep and radial cortical arteries. RIGHT-comparison saline injected (top) vs ferridex labeled VLA4-expressing GRPs (bottom) cells shows significantly more signal dropout (cell binding) in the bottom panels. Based on these data we concluded that intra-arterial administration of VLA4-expressing GRPs successfully delivered them across the BBB into the CNS.

### Example 5 Induction of Focal Demyelination in the Spinal Cord

After achieving an appropriate level of anesthesia mammals will be taken to CT suite in the radiology location in the Nelson basement. Using a Toshiba Aquilion 64 slice CT scanner at a rate of 39 frames per second we will identify the T 11 vertebral body. Under CT fluoroscopic guidance a 33 gauge needle will be inserted into the dorsal column on the right side just below the T 11 spinous process. 2 J.1L of the cytokine cocktail will be infused over 4 minutes. The needle will be reinserted at the same level on the left side and 2 J.1L of PBS is infused over 4 minutes. The needle will be then removed and the mammal will be woken up with monitoring. SSEPs are obtained daily for one week. Behavioral analyses will be obtained daily for one week.

### Example 6 Induction of Focal Demyelination in the Spinal Cord

One week after the procedure described in Example 5, the mammal will be brought back to the angiography suite. Using a Toshiba I series angiography device at 1024×1024 resolution the femoral artery will be cannulized and the anterior spinal artery identified. A microcatheter will be used to infuse Five hundred thousand to 2 million GRPs per mammal into each of several arteries that supply the damaged region of the CNS. The catheter and sheath will be removed, a surgiseal placed on the femoral artery and the mammal will be woken up with monitoring. SSEPs will be obtained weekly for eight weeks. Behavioral analysis will be obtained weekly for eight weeks.

### Example 7 Assay for Immunoprotection of Transplanted GRPs

In order to prevent immune rejection of GRPs in the CNS by circulating lymphocytes, anti-VLA4 therapy will be employed to prevent lymphocytic adhesion to the endothelial cell wall, the first step in the migration process from the bloodstream to the CNS. Anti-VLA4 therapy has been used successfully as immunosuppression in clinical use in diseases such as multiple sclerosis and other autoimmune diseases such as Crohn's disease. In the present experiment, anti-VLA4 therapy will be administered to the animal after the GRPs have entered the CNS.

As stem cell transplants begin to offer a unique potential to treat various illness of the CNS it is important to characterize their ability to survive in the host environment. This study explores strategies to monitor and maximize the survival of transplanted luciferase mouse GRP's by modulating host immune rejection mechanisms. In particular, we the strategies are focused on prevent ing T-cell trafficking across the blood brain barrier.

Anti-VLA4 antibody. Antibodies against VLA4 are obtained from two sources. A commercially available anti-VLA4 antibody used in patients with multiple sclerosis and other autoimmune diseases, natalizumab (Tysabri®), is obtained from the manufacturer Biogen-Idec. The second source is a rat-mouse hybridoma cell line from ATCC that produces an anti-0.4 integrin antibody named PS/2. To produce large quantities of antibodies for injection, the cells are sent to Immuno-Precise Antibodies (Victoria, BC, Canada) for antibody production by ascites followed by purification. These antibodies, as well as the commercially available anti-VLA4 antibodies are used in this experiment.

It is understood that the examples and embodiments described herein are for illustrative purposes only and that various modifications or changes in light thereof will be suggested to persons skilled in the art and are to be included within the spirit and purview of this application and scope of the appended claims.

1. A method for treating a demyelinating condition in a subject in need thereof, the method comprising administering to the subject a plurality of transplantation-competent GRPs, in which (i) the transplantation-competent GRPs were produced by contacting a plurality of GRPs with an agent that induces differentiation of GRPs into oligodendrocytes, and/or (ii) the administering is through an intra-arterial route.

2. The method of claim 1, with the limitation that the transplantation-competent GRPs overexpress VLA-4 on their surface.

3. The method of claim 2, with the limitation that the overexpression of VLA-4 is inducible or repressible.

4. A method for delivering GRPs to the central nervous system of a subject in need thereof, comprising administering to the subject a plurality of GRPs through an intra-arterial route.

5. The method of claim 4, wherein the subject is suffering from a demyelinating disease.

6. The method of claim 4, wherein the plurality of GRPs comprise GRPs that overexpress a polypeptide comprising the amino acid sequence of a VLA-4 subunit on their surface.

7. The method of claim 4, wherein the plurality of GRPs were contacted with an agent that induces differentiation of GRPs into oligodendrocytes.

8. The method of claim 4 or 5, wherein the GRPs myelinate neurons in the CNS of the subject.

9. The method of claim 5, wherein the subject is suffering from multiple sclerosis or transverse myelitis.

10. The method of claim 1, wherein the transplantation-competent GRPs overexpress chemokine receptors on their surface to enhance migration into the CNS.

11. A composition comprising a genetically modified GRP comprising an expression vector for expression of VLA-4, in which the composition is pharmaceutically acceptable for intra-arterial delivery to the central nervous system of a subject in need thereof.

12. The method of claim 11, wherein the overexpression of VLA-4 is inducible or repressible.

13. A composition comprising the genetically modified GRP of claim 11 and an agent that induces differentiation of GRPs into oligodendrocytes.

14. The genetically modified GRP of claim 11, in which the recombinant GRP was obtained by a method comprising differentiation of an embryonic stem cell.

15. A GRP comprising an exogenous VCAM-1 ligand on the cell surface or an exogenous nucleic acid encoding (a) VCAM-1 ligand or (b) a chemokine receptor.

16. The GRP of claim 15, further comprising a detectable label.

17. The GRP of claim 16, wherein the ORP comprises an exogenous nucleic comprising a promoter operably linked to an open reading frame encoding a reporter protein, and wherein expression of the reporter protein provides the detectable label.

18. A method for treating a CNS condition, comprising administering to a subject in need thereof a substantially pure population of therapeutic cells expressing an exogenous VCAM-1 ligand by an intra-arterial route.

19. The method of claim 18, wherein the therapeutic cells are therapeutic cells committed to a neuronal or glial cell fate.

20. The method of claim 18, wherein the CNS condition is a demyelinating condition.

21. A method for detecting therapeutic cells in the CNS, comprising imaging, by a non-invasive imaging technique, a region of the CNS of a subject administered the therapeutic cells by an intra-arterial route, the therapeutic cells being detectably labeled for detection by the non-invasive imaging technique and expressing an exogenous VCAM-1 ligand.

22. A method for treating a CNS condition, comprising dispersing a plurality of therapeutic cells to a plurality of separate brain regions in a subject in need thereof.

23. The method of claim 22, wherein the brain regions are separated by a distance of about 0.05% to about 50% of the width, length, or height of the subject's brain.
